(19) United States
(12) Patent Application Publication
(10) Pub. No.: US 2025/0127201 A1
(43) Pub. Date: Apr. 24, 2025

(54) LOW SUGAR LOW VISCOSITY SUGAR SYRUP

(71) Applicant: CORN PRODUCTS DEVELOPMENT, INC., WESTCHESTER, IL (US)

(72) Inventors: Matthew HENLEY, Bridgewater, NJ (US); Prima PATEL, Bridgewater, NJ (US)

(21) Appl. No.: 18/834,344

(22) PCT Filed: Jan. 10, 2023
(86) PCT No.: PCT/US2023/060346

§ 371 Date: Jul. 30, 2024

Related U.S. Application Data
(60) Provisional application No. 63/306,382, filed on Feb. 3, 2022.

(51) Int. Cl. A23L 29/30; A23L 27/30; C12P 19/02; C12P 19/14

(52) U.S. Cl. CPC A23L 29/35; A23L 27/33; C12P 19/02; C12P 19/14; C12Y 302/01001

(57) ABSTRACT

The invention provides a corn syrup, having a saccharide distribution having (i) a DP1+DP2 content of about 20 wt. % or less; and (ii) a DP60+ content of about 8 wt. % or less; relative to the total weight of saccharides. The invention further provides a method for preparing a corn syrup, said method comprising: a) Providing corn starch; b) Subjecting said corn starch to liquefaction to obtain a first hydrolysate; c) Subjecting said first hydrolysate to saccharification to obtain a second hydrolysate; d) Contacting said second hydrolysate with an alpha-amylase to obtain a third hydrolysate; and e) Processing the third hydrolysate to a corn syrup.

### I. TECHNICAL FIELD

The invention relates to low sugar syrups and methods for the production thereof.

### II. BACKGROUND

Sugar reduction has become a macro-trend across the globe as consumers are becoming more aware of the adverse health effects of added sugars. In addition, sugary drink taxes in many countries are driving beverage companies to seek ways to lower the amount of added sugar in their products. Further, governments are requiring “added sugars” to be clearly labelled. “Added sugars” in food are generally defined as simple sugars (mono and disaccharides).

Sugar syrups such as glucose syrups are used in many applications such as granola bars, cereal, ice cream, confectionery. Sugar syrup functions as a sweetener, thickener and humectant. The sugar syrup may be used as a viscous aqueous solution or as a dry powder. Glucose syrups are generally made from starch. Starch comprises polysaccharides.

During the process of preparing glucose syrups, the polysaccharides are hydrolyzed to shorter monosaccharides, disaccharides and oligosaccharides to obtain the desired average degree of polymerization (DP) and average dextrose equivalent (DE). Dextrose equivalent is a measure of the amount of reducing sugars present in a sugar product, expressed as a percentage on a dry basis relative to dextrose, which has a DE of 100.

Traditionally, glucose syrups are made by acid and/or enzymatic hydrolysis. The typical commercial product has a DE of 42. The 42 DE syrup has about half the sweetness of dextrose. 42 DE syrup with a solids level of 72.5 wt. % has a viscosity of less than 2,500 cP at 30° C. 42 DE syrup has about 33 wt. % sugar, sugar being defined as the sum of mono- and disaccharides (DP1+DP2). An example of such a traditional glucose syrup produced from enzymatic conversion is ENZOSE & 42 DE.

A common method to prepare glucose syrups is by performing a liquefaction step, wherein alpha amylase enzymes hydrolyze the starch into oligosaccharides, followed by a saccharification step, wherein the oligosaccharides are enzymatically hydrolyzed to mono- or disaccharides.

Glucose syrups of varying DE levels may be prepared, with DE typically ranging from 20-99. The higher the syrup conversion, the lower the viscosity of the product. However, with a reduction in viscosity, there is an increase in sugar.

Low sugar syrups are known, for example from CN111440834A. After the liquefaction step, enzyme conversion is performed using maltotetraase and pullulanase for 40-60 hours to obtain a saccharification liquid, which is decolorized, ion exchanged and concentrated to obtain a maltose oligosaccharide syrup. However, at a sugar concentration of 73.2%, the viscosity is still 2849.5 mPa's, and the reaction takes 40-60 hours.

WO2013116175A is directed to a reduced sugar syrup having a low viscosity. However, at DP1+2 of 15 wt. %, the viscosity at 71 wt. % dry solids was still significantly higher than a comparable sugar syrup.

High viscosity glucose syrup is problematic for many reasons: it is hard to pour and usually needs to be heated prior to use. Crusting may occur, it can be difficult to clean off of equipment, it is more difficult to mix, and most importantly results in undesirable texture (e.g. hard or brittle) in the final product.

There remains a need for low sugar syrups that can provide a good alternative to traditional sugar syrups. Particularly, there is a need for sugar syrups that have a comparable viscosity at equal solids level, as this is critical for replacement with similar performance.

There further remains a need for a process to produce low sugar syrups in an efficient manner.

### III. SUMMARY OF THE INVENTION

The present invention provides a corn syrup, having a saccharide distribution having (i) a DP1+DP2 content of 20 wt. % or less; and (ii) a DP60+ content of 8 wt. % or less; relative to the total weight of saccharides.

The present invention further provides a corn syrup, having a saccharide distribution having a DP1+DP2 content of 20 wt. % or less, relative to the total weight of saccharides; and having a viscosity (measured at a solid content of 72.5 wt. % and at 30° C.) of 2,500 cP or less.

The present invention further provides a method for preparing a corn syrup, said method comprising:
(a) Providing corn starch;
(b) Subjecting said corn starch to liquefaction to obtain a first hydrolysate;
(c) Subjecting said first hydrolysate to saccharification to obtain a second hydrolysate;
(d) Contacting said second hydrolysate with an alpha-amylase to obtain a third hydrolysate; and
(e) Processing the third hydrolysate to a corn syrup.

The method of the invention may be used to obtain the corn syrups of the invention.

### IV. BRIEF DESCRIPTION OF THE DRAWINGS

FIG. 1 shows a flow chart of a exemplary process of the invention.

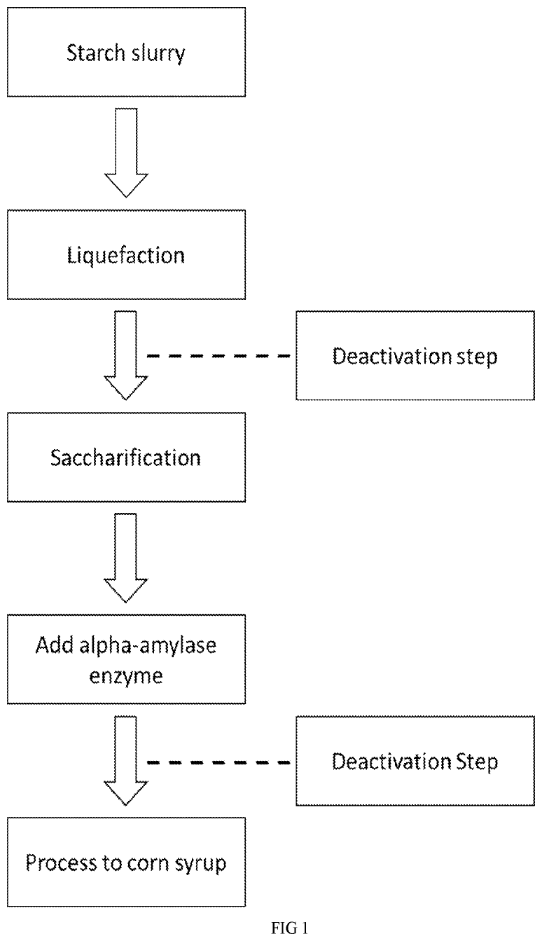

### V. DETAILED DESCRIPTION OF THE INVENTION

There is provided a corn syrup, having a saccharide distribution having (i) a DP1+DP2 content of 20 wt. % or less; and (ii) a DP60+ content of 8 wt. % or less; relative to the total weight of saccharides.

Degree of polymerization (DP) is a term known in the art. As used herein, the DP percentages refer to the weight percent of saccharides having that degree of polymerization, relative to the total weight of saccharides. The DP values thus provide the saccharide distribution of the product. DP1 refers to monosaccharides, and DP2 refers to disaccharides. Examples of monosaccharides are glucose, fructose and galactose, examples of disaccharides are lactose, maltose, and sucrose. The DP1+DP2 content refers to the sum content of DP1 and DP2 and may also be referred to as the “sugar” content. The DP60+ content refers to the sum content of polysaccharides having a degree of polymerization of 60 or more. The DP5-59 content refers to the sum content of saccharides having a degree of polymerization from 5 to 59 (5 and 59 included).

There is further provided a corn syrup, having a saccharide distribution having a DP1+DP2 content of 20 wt. % or less, relative to the total weight of saccharides; and having a viscosity (measured at a solid content of 72.5 wt. % and at 30° C.) of 2,700 cP or less.

The corn syrups preferably have a saccharide distribution having a DP1+DP2 content of 18 wt. % or less, more preferably 17 wt. % or less, even more preferably 16 wt. % or less. Preferably, the corn syrups have a saccharide distribution having a DP1+DP2 content of 10 wt. % or more, preferably 11 wt. % or more.

The corn syrups preferably have a viscosity of 2,700 cP or less, more preferably a viscosity of 2,600 cP or less, even more preferably 2,500 cP or less. Preferably, the corn syrups have a viscosity of 1,600 or more, more preferably 1,700 cP or more. The viscosity values are measured at a dry solids content of 72.5 wt. % and at 30° C. Higher viscosities can result in poor blending and pouring, and might therefore require dilution and heating. Lower viscosities can also result in poor blending.

The corn syrup according to the invention has a saccharide distribution having a DP 60+ content of 8 wt. % or less. Preferably, the corn syrup has a saccharide distribution having a DP60+ content of 7 wt. % or less, more preferably 5 wt. % or less, even more preferably 4 wt. % or less. Surprisingly, it has been found that the viscosity is improved (lowered) by reducing the DP60+ content.

Preferably, the corn syrups have a saccharide distribution having a DP1-4 content of about 70 wt. % or more. Preferably, the corn syrups have a saccharide distribution having a DP1-4 content of about 85 wt. % or less.

Preferably, the corn syrups have a saccharide distribution having a DP3 content of about 13 wt. % or more, more preferably 14 wt. % or more, even more preferably 15 wt. % or more. Preferably, the DP3 content is about 25 wt. % or less, more preferably 20 wt. % or less. DP3 saccharides are oligosaccharides, which do not count towards the sugar content of the syrup, but result in a low viscosity.

Preferably, the corn syrups have a saccharide distribution having a DP4 content of about 35 wt. % or more, more preferably about 40 wt. % or more. Preferably, the corn syrups have a saccharide distribution having a DP4 content of 60 wt. % or less, more preferably 55 wt. % or less. DP4 saccharides are oligosaccharides, which do not count towards the sugar content of the syrup, but result in a low viscosity.

Preferably, the corn syrups have a saccharide distribution wherein the DP4 fraction is the largest single degree of polymerization fraction. Thus, DP4 is the dominant fraction.

In a preferred embodiment, the co a saccharide distribution having:
a DP1+DP2 content from about 7 to about 20 wt. %, preferably from about 10 to about 17 wt. %;
a DP3 content from about 13 to about 25 wt. %, preferably from about 15 to about 20 wt. %;
a DP4 content from about 35 to about 65 wt. %, preferably from about 40 to about 55 wt. %;
a DP5-59 content from about 15 to about 30 wt. %, preferably from about 18 to about 25 wt. %; and
a DP60+ content of less than about 8 wt. %, preferably less than about 5 wt. %, for instance from about 0.5 wt. % to about 5 wt. %.

Preferably, the dextrose equivalent value (DE) of the corn syrups is about 25 to about 45, preferably about 30 to about 38, more preferably about 32 to about 36. The dextrose equivalent value is a measure of the reducing sugar content. Reducing sugars can act as reducing agents, such as in the Maillard reaction, which is important in determining the flavor of food.

The invention further provides a method for preparing a corn syrup. The method comprises step (a): providing corn starch. The corn starch may be any corn starch that is suitable for processing to a corn syrup. Such corn starch can be commercially obtained. The starch is preferably provided as an aqueous mixture of starch in water.

The method of the invention comprises step (b): subjecting said corn starch to liquefaction to obtain a first hydrolysate. Liquefaction is a common step in preparing a corn syrup, wherein the starch molecules are hydrolyzed into smaller polysaccharides. Liquefaction may be performed using a suitable acid. Preferably, liquefaction is performed enzymatically. Liquefaction of starch as such is well known in the art. Exemplary liquefaction processes and exemplary enzymes which may be used therein are e.g. described in W. Douglas Crabb and Colin Mitchinson, Enzymes used in the Processing of Starch to Sugars, Tibtech (vol. 15), September 2017 and in Starch: Chemistry and Technology 2^{nd} Addition 1984. Chapter IV. Enzymes in the Hydrolysis and Synthesis of Starch, Elsevier, Whistler et al. ed.

Preferably, step (b) is performed until a DE of 6-12, preferably a DE of 8-10, is obtained. It has been found that this allows for a suitable starting point for the following saccharification step, since it facilitates achieving a preferred distribution of saccharides in and viscosity of the final product.

Preferably, step (b) comprises adding a liquefaction alpha-amylase. As used herein the term liquefaction alpha-amylase refers to the alpha amylase used in the liquefaction step (b). The alpha-amylase is capable of hydrolyzing alpha bonds of large alpha-linked polysaccharides. Any alpha-amylase suitable for liquefaction may be used. Alpha-amylases suitable for liquefaction are well known in the art. More preferably, the liquefaction alpha-amylase is a thermostable alpha-amylase. Such thermostable alpha-amylases can be commercially obtained. Preferably, the liquefaction alpha-amylase is a Bacillus licheniformis alpha-amylase, or a variant thereof. A preferred commercial alpha-amylase is Liquozyme Supra.

The liquefaction alpha-amylase is preferably added in an amount of 0.05 to 0.80 kg enzyme per metric ton dry solids (kg/MTDS), preferably 0.10 to 0.50 kg/MTDS. Higher amounts of enzyme may be more effective and thus require less time. However, too high amounts of enzyme are less cost-efficient and may result in less process control.

Preferably, in step (b) the corn starch is present as an aqueous mixture 25-40 wt. % dry starch in water, more preferably 30-35 wt. %. Higher amounts of dry starch may be too viscous for liquefaction, whereas lower amounts are not volume efficient.

Preferably, step (b) is performed at a temperature of 85° C. to 115° C., more preferably 105° C. to 115° C. Processing at this temperature results in a more effective hydrolyzation. Because of these increased temperatures, the liquefaction alpha-amylase is preferably a thermostable alpha-amylase, such that it does not lose activity at the increased temperature.

Preferably, step (b) is performed at a pressure of 1 to 2 bar, and/or under saturated steam conditions. The pressure required for saturated steam at a given temperature may be determined using saturated steam tables.

Preferably, step (b) is performed under a high shear conditions. High shear conditions allow efficient mixing of the starch slurry and result in more efficient liquefaction. More preferably, a steam jet cooker is used. In a steam jet cooker, steam is injected through nozzles, jets, or other spargers to raise the temperature of the slurry and cook the starch.

Preferably, step (b) is performed at a pH of 4.5-6.0, more preferably 5.0 to 5.5. The pH range is preferably selected such that the liquefaction alpha-amylase is sufficiently active.

Preferably, step (b) is performed for 10 minutes to 30 minutes, more preferably 12 minutes to 20 minutes. The skilled person will understand that depending on process conditions such as enzyme dosage and activity, pressure, pH and temperature, the process may require a longer time or a shorter time. For example, increasing the enzyme concentration may allow the process to take less time.

Preferably, in the embodiments wherein step (b) comprises adding a liquefaction alpha-amylase, after step (b) but before step (c), the first hydrolysate is heated to a temperature sufficient to deactivate the liquefaction alpha-amylase; and/or brought to a pH sufficient to deactivate the liquefaction alpha-amylase, for a duration sufficient to deactivate the liquefaction alpha-amylase. Preferably, the temperature is 50° C. to 100° C., more preferably 60° C. to 70° C. Preferably, the pH is 2.0 to 3.0, more preferably 2.4 to 2.8. Preferably, said duration is at least 20 minutes, more preferably at least 30 minutes. More preferably, the temperature is 60° C. to 70° C. and the pH is 2.4 to 2.8, and the duration is at least 30 minutes. A higher pH results in less corrosion of the equipment.

The method of the invention comprises step (c): subjecting said first hydrolysate to saccharification to obtain a second hydrolysate. Saccharification is a commonly known step in preparing a corn syrup, wherein the liquefied product is further hydrolyzed to saccharides having a lower degree of polymerization. Exemplary saccharification processes and exemplary enzymes which may be used therein are e.g. described in W. Douglas Crabb and Colin Mitchinson, Enzymes used in the Processing of Starch to Sugars. Tibtech (vol. 15), September 2017 and in Starch: Chemistry and Technology 2nd Addition 1984. Chapter XXI, Glucose and fructose Containing Sweeteners from Starch, Elsevier, Whistler et al. ed.

Preferably, step (c) comprises adding a maltotetraose specific alpha-amylase, and a debranching enzyme.

A maltotetraose specific alpha-amylase is an alpha-amylase which hydrolyzes alpha bonds such that maltotetraose is formed. Maltotetraose is an oligosaccharide with a DP of 4. This facilitates keeping the DP1 or DP2 saccharides relatively low and within the preferred limits.

Preferably, the maltotetraose forming alpha-amylase is a glucan 1,4-alpha-maltotetraohydrolase (EC3.2.1.60). Such enzymes are known in the art. An exemplary suitable enzyme may be commercially obtained under the tradename Optimalt® 4G. US 2012/0135466 discloses exemplary maltotetraose forming alpha-amylases (and debranching enzymes) which may be used. Exemplary maltotetraose forming alpha-amylases which may be used are also described in https://www.brenda-enzymes.org/literature.php?e=3.2.1.60&r=732251.

Preferably, the maltotetraose specific alpha-amylase is added in an amount of 0.5 to 5 kg/MTDS, preferably from 1 to 4 kg/MTDS.

A debranching enzyme converts a branched polysaccharide into a linear one. This facilitates the linear polysaccharide to be further cleaved by the maltotetraose specific alpha-amylase. Debranching enzymes are known in the art and are for instance described in Starch: Chemistry and Technology 2^{nd} Addition 1984. Chapter XXI, Glucose and fructose Containing Sweeteners from Starch, Elsevier, Whistler et al. ed.

Preferably, the debranching enzyme is a pullulanase, more preferably a Bacillus deramificans pullulanase or a variant thereof. Such an enzyme may be commercially obtained under the tradename Optimax® L 1000, Optimax® L 2500.

Preferably, the debranching enzyme is added in an amount of 0.5 to 5 kg/MTDS, preferably from 1 to 4 kg/MTDS.

Preferably, step (c) is performed at a temperature of 50 to 80° C., preferably 60 to 70° C.

Preferably, step (c) is performed at a pH of 4.5 to 6.5, preferably 4.0 to 6.0. This allows the enzymes to be effective.

Preferably, step (c) is performed for 0.5 hour to 10 hours, more preferably 1 hour to 4 hours, even more preferably 1.5 hour to 2.5 hour.

In some embodiments, after step (c) but before step (d), the saccharification enzymes are deactivated. Thus, preferably the second hydrolysate is heated to a temperature sufficient to deactivate (i) the maltotetraose forming alpha-amylase and (ii) the debranching enzyme; and/or brought to a pH sufficient to deactivate (i) the maltotetraose forming alpha-amylase and (ii) the debranching enzyme, for a duration sufficient to deactivate (i) the maltotetraose forming alpha-amylase and (ii) the debranching enzyme. Preferably, the temperature is 50° C. to 100° C., more preferably 60° C. to 70° C. Preferably, the pH is less than 4.5, more preferably less than 4.0. Preferably, the pH is above 2.0, more preferably above 2.5, even more preferably above 3.0. Preferably, said duration is at least 20 minutes, more preferably at least 30 minutes. More preferably, the temperature is 60° C. to 70° C. and the pH is 3.0 to 4.0, and the duration is at least 30 minutes. A higher pH results in less corrosion of the equipment. This enzyme deactivation step allows to further reduce the amount of DP1 and DP2 in the final product.

The method comprises step (d): contacting said second hydrolysate with an alpha-amylase to obtain a third hydrolysate.

Preferably, step (d) is performed when a DE of 22-30, preferably a DE of 24-28, is obtained in the second hydrolysate. It was found that this facilitates achieving the desired distribution of polysaccharides.

Preferably, step (d) is performed while the saccharification enzymes are still active. This allows the process to be more efficient.

Preferably, step (d) is performed until a DE of 30-38, preferably a DE of 32-36, is obtained.

Preferably, the alpha-amylase in step (d) is a thermostable alpha-amylase. More preferably, the alpha-amylase is a Cytophaga sp. alpha-amylase or a variant thereof.

Alpha-amylase from Cytophaga sp. (herein also referred to as, “CspAmy2 amylase”), was previously described by Jeang, C-L et al. ((2002) Applied and Environmental Microbiology, 68:3651-54). The amino acid sequence of the mature form of the CspAmy2 a-amylase polypeptide is shown below as SEQ ID NO: 1:

AATNGTMMQY FEWYVPNDGQ QWNRLRTDAP YLSSVGITAV
WTPPAYKGTS QADVGYGPYD LYDLGEFNQK GTVRTKYGTK
GELKSAVNTL HSNGIQVYGD VVMNHKAGAD YTENVTAVEV
NPSNRNQETS GEYNIQAWTG FNFPGRGTTY SNFKWQWFHF
DGTDWDQSRS LSRIFKERGT GKAWDWEVSS ENGNYDYLMY
ADIDYDHPDV VNEMKKWGVW YANEVGLDGY RLDAVKHIKF
SFLKDWVDNA RAATGKEMFT VGEYWQNDLG ALNNYLAKVN
YNQSLFDAPL HYNFYAASTG GGYYDMRNIL NNTLVASNPT
KAVTLVENHD TQPGQSLEST VQPWFKPLAY AFILTRSGGY
PSVFYGDMYG TKGTTTREIP ALKSKIEPLL KARKDYAYGT
QRDYIDNPDV IGWTREGDST KAKSGLATVI TDGPGGSKRM
YVGTSNAGEI WYDLTGNRTD KITIGSDGYA TFPVNGGSVS
VWVQQ

As used herein a variant of alpha-amylase from Cytophaga sp refers to an amylase having a defined degree of amino acid sequence homology/identity to SEQ ID NO:1, for example at least 60% amino acid sequence homology/identity. For instance a variant may have at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% amino acid sequence homology/identity to SEQ ID NO:1.

More preferably, the alpha-amylase used in step (d) is a variant described in WO 2014/164777 or WO 2014/164834. The contents of these publications are herewith incorporated by reference. Preferably, the alpha amylase used in step (d) is an alpha amylase according to any one of claims 1 to 21 as published in WO 2014/164777 and/or alpha amylase according to any one claims 1 to 17 as published in WO 2014/164834. More preferably, the alpha-amylase used in step (d) is an alpha-amylase according to claim 11 as published in WO 2014/164834 or an alpha-amylase according claim 2, preferably in combination with claim 3, as published in WO 2014/164777. Preferably, the alpha amylase used in step (d) is a variant disclosed in paragraph of WO 2014/164777, most preferably CspAmy2-vC16F as disclosed in WO 2014/164777, paragraph (using SEQ ID NO: 1 for numbering): N126Y+F153W+T180H+I203Y+S241Q (i.e., CspAmy2-vC16F):

A variant thereof may also be used. For example, the above combinations of mutation are also contemplated for use in conjunction with deletions at positions corresponding to R178, G179, T180, and/or G181. Such deletions may be naturally occurring, as in the case of Bacillus licheniformis alpha-amylase.

In a preferred embodiment, the alpha-amylase used in step (d) is variant CspAmy2-vC16F as disclosed in WO 2014/164777, paragraph (using SEQ ID NO: 1 for numbering), with deletions at positions corresponding to R178 and G179, which is shown below as SEQ ID NO: 2

AATNGTMMQY FEWYVPNDGQ QWNRLRTDAP YLSSVGITAV
WTPPAYKGTS QADVGYGPYD LYDLGEFNQK GTVRTKYGTK
GELKSAVNTL HSNGIQVYGD VVMNHKAGAD YTENVTAVEV
NPSNRYQETS GEYNIQAWTG FNFPGRGTTY SNWKWQWFHF
DGTDWDQSRS LSRIFKE H GKAWDWEVSS ENGNYDYLMY
ADYDYDHPDV VNEMKKWGVW YANEVGLDGY RLDAVKHIKF
QFLKDWVDNA RAATGKEMFT VGEYWQNDLG ALNNYLAKVN
YNQSLFDAPL HYNFYAASTG GGYYDMRNIL NNTLVASNPT
KAVTLVENHD TQPGQSLEST VQPWFKPLAY AFILTRSGGY
PSVFYGDMYG TKGTTTREIP ALKSKIEPLL KARKDYAYGT
QRDYIDNPDV IGWTREGDST KAKSGLATVI TDGPGGSKRM
YVGTSNAGEI WYDLTGNRTD KITIGSDGYA TFPVNGGSVS
VWVQQ

In SEQ ID NO: 2, N126Y, F153W, T180H, 1203Y, and S241Q, are underlined.

GRAS notice 617 provides a further description of an alpha-amylase which may advantageously be used in step (d).

A preferred alpha-amylase for use in step (d) may be obtained commercially under the name GC127 enzyme from Genencor. It has been found that the preferred alpha-amylases facilitate achieving the desired saccharide distribution.

Preferably, in step (d) the alpha-amylase is added in an amount of 0.30 to 2.0 kg/MTDS. More preferably, the alpha-amylase is added in an amount of 0.40 to 0.60 kg/MTDS.

Preferably, step (d) is performed at a temperature of 50 to 80° C., preferably 60 to 70° C.:

Preferably, step (d) is performed for 0.5 hour to 3 hours, preferably 1 hour to 2.5 hours.

Preferably, after step (d), the third hydrolysate is heated to a temperature sufficient to deactivate the step (d) alpha-amylase and optionally the saccharification enzymes, and/or brought to a pH sufficient to deactivate the step (d) alpha-amylase and optionally the saccharification enzymes, for a duration sufficient to deactivate the step (d) alpha-amylase and optionally the saccharification enzymes. Preferably, the temperature is 50° C. to 100° C., preferably 60° C. to 70° C. Preferably, the pH is less than 4.5, more preferably less than 4.0. Preferably the pH is more than 2.0, more preferably more than 2.5, even more preferably more than 3.0. A higher pH results in less corrosion of the equipment. Preferably, said duration is at least 20 minutes, more preferably at least 30 minutes, more preferably at least 1 hour. More preferably, the temperature is 60° C. to 80° C. and the pH is 3.0-4.0, for a duration of at least 30 minutes.

Preferably, after the deactivation using temperature and/or pH, the third hydrolysate is cooled and left to stand. Preferably, the third hydrolysate is left to stand for at least 4 hours, more preferably at least 6 hours, even more preferably at least 12 hours. This allows the solids, comprising the enzymes and remaining starch, to sink, which facilitates the processing step.

The method comprises step (e): processing the third hydrolysate to a corn syrup. Said processing may be performed according to any method known in the art. Such methods may include removal of insoluble proteins by filtration or centrifuges. These methods may be followed by carbon treatment and/or ion exchange or polishing resins to remove soluble proteins, enzymes, ash, and flavor or color components, and evaporation to remove water.

Preferably, step (e) comprises filtering or centrifuging the third hydrolysate to remove insoluble materials.

Preferably, step (e) comprises contacting the third hydrolysate with an ion-exchange resin, a polishing resin, and/or active carbon. This allows to immobilize the enzymes, both soluble and insoluble, in the hydrolysate and thus separate these from the corn syrup. Thus, no deactivation step using temperature/pH is required. It further allows to remove further protein material, ash, flavor and/or color components, thereby decolorizing and purifying the corn syrup.

Preferably, step (e) comprises a dehydrating the product to 70-85 wt. % dry solids. Said dehydrating is preferably performed using heat, optionally in combination with sub-atmospheric pressure. Dehydration results in a product which has desired flow properties, while saving space and weight in transport.

### VI. EXAMPLES

### General Procedure Experiments A-D and Examples 1-10

30-35 wt. % dry solids native corn starch slurry was prepared in tap water. The pH was adjusted between 5.0-5.5 using hydrochloric acid and/or sodium hydroxide. Liquozyme® Supra (2.2×) (liquid alpha amylase for liquefaction from Novozymes, a variant of a Bacillus licheniformis alpha-amylase) was added to the slurry at a dose of 0.25 kg/MTDS and steam jet cooked at 93° C. for complete liquefaction. At dextrose equivalent (DE) 6-14, Liquozyme® Supra was deactivated by adjusting the pH to 2.4-2.8 using hydrochloric acid, and holding at this pH at 65° C. for more than 30 minutes. The pH of the sample was readjusted between 5.0-6.0 and of Optimalt® 4G (maltotetraose forming alpha-amylase from DuPont) at a dose of 1.5 kg/MTDS, and debranching enzyme Optimax® L 1000 (pullulanase from DuPont, variant of a Bacillus deramificans pullulanase expressed in Baciullus licheniformis) at a dose of 1.5 kg/MTDS were added. Between DE 26-30 the reaction was terminated via ion exchange/filtration to remove processing aid(s) and byproducts. The pH of the sample was adjusted to between 5.0-6.0. Enzyme GC127 (obtained from Genencor) was added to the sample at 0.47 kg/MTDS. When sample DE was between 32-37, reaction was terminated by acid deactivation (pH below 4) or the enzyme was removed via ion exchange.

The sample was then filtered using earth diatomite (Kenite 5200) to remove protein and other undigested material. Sample was dehydrated using a roto-evaporator under vacuum and heat (60° C.) until dry solids were above 75 wt. %.

The final product was characterized for sugar content, molecular weight distribution, dextrose equivalence and viscosity. Sugar content was analyzed using gel permeation chromatography (GPC) equipped with TOSOH BIOSCIENCE TSK 2500 columns for Degree of polymerization (DP) 1-4 and 60+ analysis and molecular weight distribution. Viscosity measurement was conducted at 72.5 wt. % dry solids (using Karl-Fischer titration), measured using Anton Parr RheolabOC equipped with spindle CC27, with a shear rate of 10/s, 10 minute hold at 30° C. Dextrose equivalence (D.E.) of samples was determined using an osmometer (Advanced Instruments), where sample was analyzed at 15 wt. % dry solids using D.I. water. The following calculation was used to determine D.E.: Osmometer reading (mOsm)×0.09837+2.59.

### Reference Experiments A-D

Tables 1 and 2: Comparative experiments. Liquozyme Supra was deactivated at various DE, after which Optimalt 4G and Optimax L 1000 were added. The reaction was stopped at various DE.

| Ref. | Liquozyme Supra | Reaction | Viscosity (72.5 wt. % | Final |
|---|---|---|---|---|
| Exp. | deactivated at | stopped at | solids, 30° C.)/cP | DE |
| A | DE 8.0 | DE 28 | 4,728 | 29.2 |
| B | DE 9.4 | DE 29 | 9,819 | 28.4 |
| C | DE 10.5 | DE 27 | — | 26.1 |
| D | DE 11.6 | DE 30 | 7,479 | 30.4 |

| | Saccharide distribution | | | | | | | | |
|---|---|---|---|---|---|---|---|---|---|
| Ref. | DP1/ | DP2/ | DP3/ | DP4/ | DP1 + 2/ | DP3 + 4/ | DP 1-4/ | DP 5-59/ | DP60+/ |
| Exp. | wt. % | wt. % | wt. % | wt. % | wt. % | wt. % | wt. % | wt. % | wt. % |
| A | 3.45 | 6.44 | 11.50 | 54.53 | 9.89 | 66.03 | 75.92 | 16.02 | 8.25 |
| B | 2.72 | 6.20 | 11.65 | 48.37 | 8.92 | 60.02 | 68.94 | 15.91 | 15.07 |
| C | 1.92 | 5.39 | 10.31 | 42.75 | 7.31 | 53.06 | 60.37 | 18.38 | 21.38 |
| D | 3.53 | 7.74 | 13.51 | 50.99 | 11.27 | 64.50 | 75.77 | 16.13 | 14.24 |

From the above table, it can be observed that the viscosity of the syrups was above 2,500 cP (72.5 wt. % solids, 30° C.)

### Examples 1-3

Tables 3 and 4: Liquozyme Supra was deactivated at DE 8.0. Alpha-amylase (GC 127) was added at DE 28. The reaction was stopped at various DE.

| | Reaction | Viscosity (72.5 wt. % solids, | |
|---|---|---|---|
| Example | stopped at | 30° C.)/cP | Final DE |
| 1 | DE 32 | 2,354 | 33.5 |
| 2 | DE 34 | 2,113 | 35.5 |
| 3 | DE 36 | 2,203 | 38.8 |

| | Saccharide distribution | | | | | | | | |
|---|---|---|---|---|---|---|---|---|---|
| | DP1/ | DP2/ | DP3/ | DP4/ | DP1 + 2/ | DP3 + 4/ | DP 1-4/ | DP 5-59/ | DP60+/ |
| Example | wt. % | wt. % | wt. % | wt. % | wt. % | wt. % | wt. % | wt. % | wt. % |
| 1 | 4.58 | 7.90 | 15.09 | 50.01 | 12.48 | 65.10 | 77.58 | 20.60 | 1.89 |
| 2 | 5.58 | 9.02 | 16.11 | 48.88 | 14.60 | 64.99 | 79.59 | 19.48 | 1.06 |
| 3 | 6.22 | 9.61 | 18.07 | 47.44 | 15.83 | 65.51 | 81.34 | 18.35 | 0.62 |

The addition of the final alpha-amylase at DE 38 resulted in a decrease of viscosity from 4,728 cP to below 2,500 cP, compared to reference experiment A. The final DP1+2 content was below 17 wt. % of total saccharides. The final DP1-4 content was above 77 wt. % of total saccharides.

### Examples 4,5

Tables 5 and 6: Liquozyme Supra was deactivated at DE 9.4, Alpha-amylase (GC 127) was added at DE 29. The reaction was stopped at various DE.

| | Reaction | Viscosity (72.5 wt. % solids, | |
|---|---|---|---|
| Example | stopped at | 30° C.)/cP | Final DE |
| 4 | DE 34 | 2,299 | 34.0 |
| 5 | DE 35 | 1,938 | 35.2 |

| | Saccharide distribution | | | | | | | | |
|---|---|---|---|---|---|---|---|---|---|
| | DP1/ | DP2/ | DP3/ | DP4/ | DP1 + 2/ | DP3 + 4/ | DP 1-4/ | DP 5-59/ | DP60+/ |
| Example | wt. % | wt. % | wt. % | wt. % | wt. % | wt. % | wt. % | wt. % | wt. % |
| 4 | 4.32 | 8.36 | 16.77 | 46.73 | 12.68 | 63.50 | 76.18 | 21.37 | 3.67 |
| 5 | 4.83 | 9.04 | 17.52 | 46.21 | 13.87 | 63.73 | 77.60 | 20.61 | 2.61 |

The addition of the final alpha-amylase at DE29 resulted in a decrease of viscosity from 7,479 cP to below 2,300 cP, compared to reference experiment B. The final DP1+2 content was below 14 wt. % of total saccharides. The final DP1-4 content was above 76 wt. % of total saccharides.

### Example 6

Tables 7 and 8: Liquozyme Supra was deactivated at DE 9.5. Alpha-amylase (GC 127) was added at DE 27. The reaction was stopped at DE 36.

| | Reaction | Viscosity (72.5 wt. % solids, | |
|---|---|---|---|
| | stopped at | 30° C.)/cP | Final DE |
| 6 | DE 36 | 2.025 | 34.6 |

| | Saccharide distribution | | | | | | | | |
|---|---|---|---|---|---|---|---|---|---|
| | DP1/ | DP2/ | DP3/ | DP4/ | DP1 + 2/ | DP3 + 4/ | DP 1-4/ | DP 5-59/ | DP60+/ |
| | wt. % | wt. % | wt. % | wt. % | wt. % | wt. % | wt. % | wt. % | wt. % |
| 6 | 4.52 | 8.60 | 17.14 | 44.92 | 13.12 | 62.06 | 75.18 | 22.30 | 3.24 |

The final DP1+2 content was below 14 wt. % of total saccharides. The final DP1-4 content was above 75 wt. % of total saccharides.

### Examples 7-10

Tables 9 and 10: Liquozyme Supra was deactivated at DE 10.5, Alpha-amylase (GC 127) was added at DE 27. The reaction was stopped at various DE.

| | Reaction | Viscosity (72.5 wt. % solids, | |
|---|---|---|---|
| Example | stopped at | 30° C.)/cP | Final DE |
| 7 | DE 35 | 2,087 | 33.9 |
| 8 | DE 41 | 2,205 | 35.2 |
| 9 | DE 37 | 1,562 | 35.7 |
| 10 | DE 39 | 1,731 | 38.3 |

| | Saccharide distribution | | | | | | | | |
|---|---|---|---|---|---|---|---|---|---|
| | DP1/ | DP2/ | DP3/ | DP4/ | DP1 + 2/ | DP3 + 4/ | DP 1-4/ | DP 5-59/ | DP60+/ |
| Example | wt. % | wt. % | wt. % | wt. % | wt. % | wt. % | wt. % | wt. % | wt. % |
| 7 | 3.97 | 8.45 | 17.04 | 42.89 | 12.42 | 59.93 | 72.35 | 23.75 | 4.68 |
| 8 | 4.39 | 8.94 | 17.81 | 43.00 | 13.33 | 60.81 | 74.14 | 22.99 | 3.82 |
| 9 | 4.87 | 9.39 | 18.48 | 42.83 | 14.26 | 61.31 | 75.57 | 22.29 | 3.16 |
| 10 | 5.29 | 9.86 | 19.20 | 42.13 | 15.15 | 61.33 | 76.48 | 21.07 | 2.93 |

The final DP1+2 content was below 16 wt. % of total saccharides. The final DP1-4 content was above 72 wt. % of total saccharides.

### Example 11

Table 11 and 12: Liquozyme Supra was deactivated at DE 11.6, Alpha-amylase (GC 127) was added at DE 30. The reaction was stopped at DE 37.

| Example | Reaction stopped at | Viscosity/cP | Final DE |
|---|---|---|---|
| 11 | DE 37 | 1,822 | 36.7 |

| | Saccharide distribution | | | | | | | | |
|---|---|---|---|---|---|---|---|---|---|
| | DP1/ | DP2/ | DP3/ | DP4/ | DP1 + 2/ | DP3 + 4/ | DP 1-4/ | DP 5-59/ | DP60+/ |
| Example | wt. % | wt. % | wt. % | wt. % | wt. % | wt. % | wt. % | wt. % | wt. % |
| 11 | 5.12 | 9.56 | 18.03 | 45.75 | 14.68 | 63.78 | 78.46 | 19.87 | 2.47 |

The addition of the final alpha-amylase at DE 30 resulted in a decrease of viscosity from 7,479 cP to below 1,900 cP, compared to Reference Experiment D. The final DP1+2 content was below 17 wt. % of total saccharides. The final DP1-4 content was above 78 wt. % of total saccharides.

### Examples 12 and 13

For example 12, 30-35 wt. % dry solids native corn starch slurry was prepared in tap water. The pH was adjusted between 5.0-5.5 using hydrochloric acid and/or sodium hydroxide. Liquozyme® Supra (2.2×) (liquid alpha amylase for liquefaction from Novozymes, a variant of a Bacillus licheniformis alpha-amylase) was added to the slurry at a dose of 0.25 kg/MTDS and steam jet cooked at 98° C. until the liquefact was hydrolysed to 8.2 DE. The Liquozyme® Supra was deactivated by adjusting the pH to 2.4-2.8 using hydrochloric acid, and holding at this pH at 65° C. for 12 minutes. The final DE after this step was determined to be 8.9.

The pH of the first hydrolysate was readjusted between 5.0-6.0 and of Optimalt® 4G (maltotetraose forming alpha-amylase from DuPont) at a dose of 4.5 kg/MTDS, and debranching enzyme Optimax® L 1000 (pullulanase from DuPont, variant of a Bacillus deramificans pullulanase expressed in Baciullus licheniformis) at a dose of 4.5 kg/MTDS were added. This reaction was performed for 3 hours, at which point the DE was 26.3.

The pH of the second hydrolysate was adjusted to between 5.0-6.0. Enzyme GC127 (obtained from Genencor) was added to the sample at 1.41 kg/MTDS. The reaction was continued for a further hour, at which point the DE was 32.6.

The reaction was terminated by adjusting the pH to 2.4-2.8 using hydrochloric acid, and holding at this pH at 65° C. for 2.5 hours. The final DE was 33.4.

The product was chilled overnight, clarified and blended. A blended sample was characterized for sugar content, molecular weight distribution, dextrose equivalence and viscosity. Sugar content was analyzed using GPC equipped with BioRad Aminex HPX-42A column. Viscosity measurement was conducted at 72.5 wt. % dry solids (using Karl-Fischer titration), measured using Anton Parr RheolabOC equipped with spindle CC27, with a shear rate of 10/s, 10 minute hold at 30° C. Dextrose equivalence (D.E.) of samples was determined using an osmometer (Advanced Instruments), where sample was analyzed at 15 wt. % dry solids using D.I. water. The following calculation was used to determine D.E.: Osmometer reading (mOsm)×0.09837+2.59.

For Example 13, 30-35 wt. % dry solids native corn starch slurry was prepared in tap water. The pH was adjusted between 5.0-5.5 using hydrochloric acid and/or sodium hydroxide. Liquozyme® Supra (2.2×) (liquid alpha amylase for liquefaction from Novozymes, a variant of a Bacillus licheniformis alpha-amylase) was added to the slurry at a dose of 0.75 kg/MTDS and steam jet cooked at 98° C. until the liquefact was hydrolysed to 9.2 DE. The Liquozyme® Supra was deactivated by adjusting the pH to 2.4-2.8 using hydrochloric acid, and holding at this pH at 65° C. for 20 minutes. The final DE after this step was determined to be 10.4.

The pH of the first hydrolysate was readjusted between 5.0-6.0 and of Optimalt® 4G (maltotetraose forming alpha-amylase from DuPont) at a dose of 4.5 kg/MTDS, and debranching enzyme Optimax® L 1000 (pullulanase from DuPont, variant of a Bacillus deramificans pullulanase expressed in Baciullus licheniformis) at a dose of 4.5 kg/MTDS were added. This reaction was performed for 3 hours, at which point the DE was 25.1.

The pH of the second hydrolysate was adjusted to between 5.0-6.0. Enzyme GC127 (obtained from Genencor) was added to the sample at 1.41 kg/MTDS. The reaction was continued for a further hour, at which point the DE was 34.1.

The reaction was terminated by adjusting the pH to 2.4-2.8 using hydrochloric acid, and holding at this pH at 65° C. for 2.5 hours. The final DE was 35.1.

The product was chilled overnight, clarified and blended. A blended sample was characterized for sugar content, molecular weight distribution, dextrose equivalence and viscosity.

TABLE 13
sugar profile, viscosity and final DE of pilot plant trials

| | Saccharide distribution | | | | | | | | |
|---|---|---|---|---|---|---|---|---|---|
| | DP1/ | DP2/ | DP3/ | DP4/ | DP1 + 2/ | DP1-4/ | DP11+/ | Viscosity/ | final |
| Ex. | wt. % | wt. % | wt. % | wt. % | wt. % | wt. % | wt. % | cP | DE |
| 12 | 5.72 | 8.65 | 15.51 | 49.25 | 14.37 | 79.13 | 7.91 | 2,500 | 34.3 |
| 13 | 5.98 | 8.93 | 15.57 | 45.86 | 14.91 | 76.34 | 12.57 | 2,450 | 35.1 |

The final DP1+2 content was below 15 wt. %. The viscosity was around 2,500 cP. The final DP1-4 content was above 76 wt. %.

While the principles of the invention have been set out above in connection with specific embodiments, it is to be understood that this description is merely made by way of example and not as a limitation of the scope of protection, which is determined by the appended claims.

1. A corn syrup, having a saccharide distribution having (i) a DP1+DP2 content of about 20 wt. % or less; and (ii) a DP60+ content of about 8 wt. % or less; relative to the total weight of saccharides.

2. A corn syrup according to claim 1, having a saccharide distribution having a DP1+DP2 content of about 20 wt. % or less, relative to the total weight of saccharides; and having a viscosity (measured at a solid content of 72.5 wt. % and at 30° C.) of about 2,700 cP or less.

3. The corn syrup according to claim 1, having a saccharide distribution having a DP1+DP2 content of about 18 wt. % or less, preferably about 17 wt. % or less.

4. The corn syrup according to claim 1, having a saccharide distribution having a DP1+DP2 content of about 10 wt. % or more, preferably about 11 wt. % or more.

5. The corn syrup according to claim 1, having a viscosity of about 2,700 cP or less, preferably having a viscosity of about 2,600 cP or less, wherein viscosity is measured at a solid content of 72.5 wt. % and 30° C.

6. The corn syrup according to claim 1, having a viscosity of about 1,600 or more, preferably about 1,700 cP or more, wherein viscosity is measured at a solid content of 72.5 wt. % and 30° C.

7. The corn syrup according to claim 1, having a saccharide distribution having a DP60+ content of about 8 wt. % or less, preferably about 7 wt. % or less.

8. The corn syrup according to claim 1, having a saccharide distribution having a DP60+ content of about 6 wt. % or less, preferably about 5 wt. % or less, more preferably about 4 wt. % or less.

9. The corn syrup according to claim 1, having a saccharide distribution having a DP1-4 content of about 70 wt. % or more.

10. The corn syrup according to claim 1, having a saccharide distribution having a DP3 content of about 13 wt. % or more, preferably about 14 wt. % or more, preferably about 15 wt. % or more.

11. The corn syrup according to claim 1, having a saccharide distribution having a DP4 content of about 35 wt. % or more, preferably about 40 wt. % or more.

12. The corn syrup according to claim 1, having a saccharide distribution having a DP4 content of about 60 wt. % or less, preferably about 55 wt. % or less.

13. The corn syrup according to claim 1, having a saccharide distribution wherein the DP4 fraction is the largest single degree of polymerization fraction.

14. The corn syrup according to claim 1, having a saccharide distribution having:
a DP1+DP2 content from about 7 to about 20 wt. %, preferably from about 10 to about 17 wt. %;
a DP3 content from about 13 to about 25 wt. %, preferably from about 15 to about 20 wt. %;
a DP4 content from about 35 to about 65 wt. %, preferably from about 40 to about 55 wt. %;
a DP5-59 content from about 15 to about 30 wt. %, preferably from about 18 to about 25 wt. %; and
a DP60+ content of less than about 8 wt. %, preferably less than about 5 wt. %, for instance from about 0.5 wt. % to about 5 wt. %.

15. The corn syrup according to claim 1, wherein the dextrose equivalent value (DE) is from about 30 to about 40, preferably from about 31 to about 37, more preferably from about 33 to about 35.

16. A method for preparing a corn syrup, said method comprising:
a) Providing corn starch;
b) Subjecting said corn starch to liquefaction to obtain a first hydrolysate;
c) Subjecting said first hydrolysate to saccharification to obtain a second hydrolysate;
d) Contacting said second hydrolysate with an alpha-amylase to obtain a third hydrolysate; and
e) Processing the third hydrolysate to a corn syrup.

17. The method according to claim 16, wherein step (b) is performed until a DE of 6-12, preferably a DE of 8-10, is obtained.

18. The method according to claim 16, wherein step (b) comprises adding a liquefaction alpha-amylase, preferably a thermostable alpha-amylase, preferably a Bacillus licheniformis alpha-amylase or a variant thereof.

19. The method according to claim 18, wherein said liquefaction alpha-amylase is added in an amount of 0.05 to 0.80 kg per metric ton of dry solids (kg/MTDS), more preferably 0.10 to 0.50 kg/MTDS.

20. The method according to claim 16, wherein step (b):
has corn starch present as an aqueous mixture of about 25-40 wt. % dry starch in water, preferably about 30-35 wt. %;
is performed at a temperature of about 85° C. to about 115° C., preferably about 105° C. to about 115° C.;
is performed at a pressure of about 1 to about 2 bar, and/or under saturated steam conditions;
is performed under high shear conditions, preferably using a steam jet cooker;
is performed at a pH of from about 4.5 to about 6.0, preferably from about 5.0 to about 5.5; and/or
is performed for at least about 10 minutes, for instance from about 10 minutes to about 60 minutes, for instance from about 12 minutes to about 30 minutes.

21-40. (canceled)


SEQUENCE LISTING (2 sequences)

SEQ ID NO: 1 - protein, 485 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 2 - protein, 483 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRYQETSGEYNIQAWTGFNFPGRGTTYSNWKWQWFHFDGTDWDQSRSLSRIFKFHGKAWDWEVSSENGNYDYLMYADYDYDHPDVVNEMKKWGVWYANEVGLDGYRLDAVKHIKFQFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ